(19) United States
(12) Patent Application Publication
(10) Pub. No.: US 2022/0117722 A1
(43) Pub. Date: Apr. 21, 2022

(54) PERMANENT EXPANDABLE PROSTHETIC BREAST IMPLANT AND DELIVERY SYSTEM

(71) Applicant: Expa Medical Devices, San Diego, CA (US)

(72) Inventors: Benjamin Weiss, Ocean, NJ (US); Matthew Goldschmidt, San Diego, CA (US)

(21) Appl. No.: 17/499,598

(22) Filed: Oct. 12, 2021

Related U.S. Application Data
(60) Provisional application No. 63/092,234, filed on Oct. 15, 2020.

(51) Int. Cl. A61F 2/12; B29C 35/08; B29C 35/00

(52) U.S. Cl. CPC A61F 2/12; B29C 35/0805; B29L 2031/7532; A61F 2210/0085; B29C 35/002

(57) ABSTRACT

In some instances, the disclosure provides performing a breast implant procedure using a permanent expandable prosthetic breast implant. The method comprises subsequent to placing the permanent expandable prosthetic breast implant within a breast region of an individual, injecting a polymer into the permanent expandable prosthetic breast implant, and changing a viscosity of the polymer within the permanent expandable prosthetic breast implant using a chemical reaction.

### CROSS-REFERENCE TO RELATED APPLICATIONS

This patent application claims the benefit of U.S. Provisional Patent Application No. 63/092,234, filed Oct. 15, 2020, which is incorporated by reference.

### BACKGROUND

Breast implants may be commonly used to replace and/or augment an individual's breasts. For example, due to disease, it may become medically necessary to remove some or all of the mammary gland and surrounding tissues. In such cases, a breast prosthetic implant may be used to maintain or supplement the appearance of the breast after the removal. By restoring or augmenting the normal appearance of the body, there may be beneficial psychological effects for the individual.

Traditionally, prosthetic breast implants may include an elastomeric implant envelope filled with a filler material such as saline or silicone. Over the course of the breast reconstruction or augmentation process, the breast tissue may be expanded by increasing the filler material within the envelope. This may occur in two separate surgical stages. In the first stage, an operating physician may insert a breast or tissue expander within the individual's breast. The expander may take the form of an envelope or enclosure and once inserted, the expander may be progressively filled with saline injections until a desired size or volume is achieved. At such a time, the second surgical stage may occur and another invasive procedure is performed where the tissue expander is removed and replaced with a realistic-feeling, permanent implant. In other words, the breast or tissue expander inserted within the first stage is not permanent and is eventually removed for a more permanent implant. However, using two separate surgical steps may cause the individual a greater amount of pain as well as risk (both surgical and peri-operative) than necessary. Accordingly, an alternative where only one surgical procedure is performed is preferable.

Currently, there have been several solutions to create an expandable implant that avoids the second invasive surgery. For example, solutions that have been utilized for a permanent implant include creation of a gas via a chemical reaction or using pressured gas cartridges. Another example is a BECKER expander implant that includes an outer portion containing a silicone gel and an inner portion to which saline may be added. However, these examples have their draw-backs including the decreased quality of “feel” of the implant (e.g., these implants might not feel natural or might not feel similar to actual breast tissue). Additionally, in the gas examples above, they may also have possible bursting complications. As such, there remains a technical need to provide a permanent expandable prosthetic breast implant that mitigates or obviates at least some of the above-noted disadvantages.

### SUMMARY

In some examples, the present application may provide a permanent expandable prosthetic breast implant (permanent implant) and a method for expansion of the implant. For example, after an operating physician inserts the permanent implant within the individual's breast, the operating physician may inject the permanent implant with a polymer over a period of time such as a month. Once the desired size is reached, the operating physician may perform a non-invasive process that changes a characteristic of the injected polymer. For instance, the operating physician may perform a process that increases the viscosity of the polymer such that the consistency is more similar to actual body tissue.

In some instances, the disclosure provides performing a breast implant procedure using a permanent expandable prosthetic breast implant. The method comprises: subsequent to placing the permanent expandable prosthetic breast implant within a breast region of an individual, injecting a polymer into the permanent expandable prosthetic breast implant; and changing a viscosity of the polymer within the permanent expandable prosthetic breast implant using a chemical reaction.

In some examples, the polymer comprises a photopolymer, and wherein changing the viscosity of the polymer comprises: activating one or more light sources within the permanent expandable prosthetic breast implant to increase the viscosity of the photopolymer.

In some variations, the polymer is a mixture that comprises the photopolymer and at least one other material. The other materials may include, but are not limited to, crosslinking agents, photo-initiators, and/or mixtures of other silicones.

In some instances, activating the one or more light sources comprises: receiving, from a user device and by an activation device within the permanent expandable prosthetic breast implant, one or more instructions indicating to change the viscosity of the polymer; and in response to receiving the one or more instructions, providing one or more commands to the one or more light sources to emit light within the permanent expandable prosthetic breast implant.

In some examples, the activation device comprises a power source, a wireless communications interface, and one or more processors.

In some variations, the one or more light sources are inserted within the permanent expandable prosthetic breast implant subsequent to placing the permanent expandable prosthetic breast implant within the breast region of the individual.

In some instances, the activation device is inserted within the permanent expandable prosthetic breast implant subsequent to placing the permanent expandable prosthetic breast implant within the breast region of the individual.

In some examples, the one or more light sources is configured to emit light in the ultraviolet (UV) spectrum or visible spectrum.

In some variations, a permanent expandable prosthetic breast implant is provided. The permanent expandable prosthetic breast implant comprises: an exterior surface layer; an interior spacing within the exterior surface layer, wherein the interior spacing comprises a photopolymer; and one or more light sources within the exterior surface layer, wherein the one or more light sources are configured to cause a change in a characteristic of the photopolymer by emitting a light.

In some instances, the permanent expandable prosthetic breast implant further comprises: an activation device configured to: receive, from a user device, an instruction to change in the characteristic of the photopolymer; and in response to receiving the instruction, providing one or more commands to the one or more light sources to emit the light.

In some examples, the activation device comprises a power source, a wireless communications interface, and one or more processors.

In some variations, the one or more light sources are inserted within the permanent expandable prosthetic breast implant subsequent to placing the permanent expandable prosthetic breast implant within the breast region of an individual.

In some instances, the activation device is inserted within the permanent expandable prosthetic breast implant subsequent to placing the permanent expandable prosthetic breast implant within the breast region of an individual.

In some examples, the light is in the ultraviolet (UV) spectrum.

In some variations, the light is in the visible spectrum.

In some instances, a system is provided. The system comprises a permanent expandable prosthetic breast implant, comprising: an interior spacing within the exterior surface layer, wherein the interior spacing comprises a photopolymer; and one or more light sources within the exterior surface layer, wherein the one or more light sources are configured to cause a change in a characteristic of the polymer by emitting a light. The system further comprises a user device configured to provide an instruction to cause the change in the characteristic of the photopolymer.

In some examples, the permanent expandable prosthetic breast implant further comprises: an activation device configured to: receive, from the user device, the instruction to cause the change in the characteristic of the photopolymer; and in response to receiving the instruction, providing one or more commands to the one or more light sources to emit the light.

In some variations, the activation device comprises a power source, a wireless communications interface, and one or more processors.

In some instances, the light is in the ultraviolet (UV) spectrum.

In some examples, the light is in the visible spectrum.

All examples and features mentioned above may be combined in any technically possible way.

### BRIEF DESCRIPTION OF THE DRAWINGS

The subject technology will be described in even greater detail below based on the exemplary figures, but is not limited to the examples. All features described and/or illustrated herein can be used alone or combined in different combinations. The features and advantages of various examples will become apparent by reading the following detailed description with reference to the attached drawings which illustrate the following:

FIG. 1 shows an exemplary permanent implant after insertion into an individual's body in accordance with one or more examples of the present application.

FIGS. 2a and 2b are exemplary permanent implants in accordance with one or more examples of the present application.

FIG. 3 is an exemplary injector that is used to inject polymers and/or other components into the permanent implant in accordance with one or more examples of the present application.

### DETAILED DESCRIPTION

Examples of the presented application will now be described more fully hereinafter with reference to the accompanying FIGs., in which some, but not all, examples of the application are shown. Indeed, the application may be exemplified in different forms and should not be construed as limited to the examples set forth herein; rather, these examples are provided so that the application will satisfy applicable legal requirements. Where possible, any terms expressed in the singular form herein are meant to also include the plural form and vice versa, unless explicitly stated otherwise. Also, as used herein, the term “a” and/or “an” shall mean “one or more” even though the phrase “one or more” is also used herein. Furthermore, when it is said herein that something is “based on” something else, it may be based on one or more other things as well. In other words, unless expressly indicated otherwise, as used herein “based on” means “based at least in part on” or “based at least partially on”.

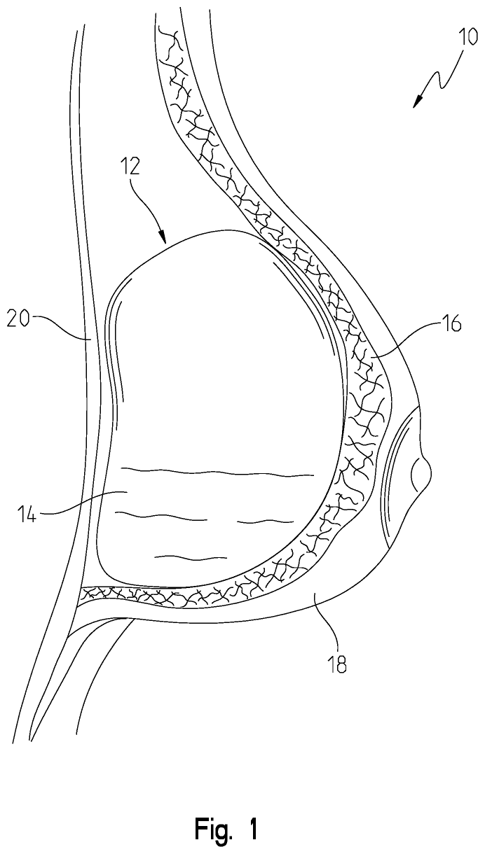

A permanent expandable prosthetic breast implant (permanent implant) and a method for expansion of the implant are herein disclosed that provide for changing a characteristic of an injected polymer within the permanent implant. FIG. 1 shows an exemplary permanent implant after insertion into an individual's body in accordance with one or more examples of the present application. In particular, FIG. 1 shows a breast region 10 of an individual's body with an inserted permanent implant 12. For example, the breast region 10 includes the pectoral muscle 16, the epidermis or skin 18, the chest wall 20. The permanent implant 12 may be inserted within the breast region 10. In some examples and as shown, the permanent implant 12 is inserted between the pectoral muscle 16 and the chest wall 20. This may be called a sub-muscular breast implant placement. In other examples, the permanent implant 12 may be a sub-glandular implant and placed behind the skin 18 and the breast gland, but in front of the pectoral muscle 16.

The permanent implant 12 may be filled with a polymer 14. The polymer 14 may be and/or include a photo-reactive initiator, and or cross-linking component, which can be used for light-based polymerization. For example, an operating physician may operate on the individual and position, insert, and/or otherwise place the permanent implant 12 within an individual's breast. Initially, right after insertion, the permanent implant 12 may be empty. In other words, there might not be any or very little polymer 14 within the permanent implant 12 at the time of insertion. At certain time increments within a set time period (e.g., within thirty days or around six weeks), the operating physician and/or another person may inject the polymer 14 into the permanent implant 12. This may continue to until the polymer 14 reaches a certain threshold amount. At such a time, the operating physician may perform a process to change a characteristic of the polymer 14 from a first characteristic value to a second characteristic value. For example, the operating physician may change (e.g., increase) a viscosity of the polymer 14 such that the consistency of the polymer 14 is more similar to that of the actual body tissue. Additionally, and/or alternatively, the operating physician may change a density of the polymer 14.

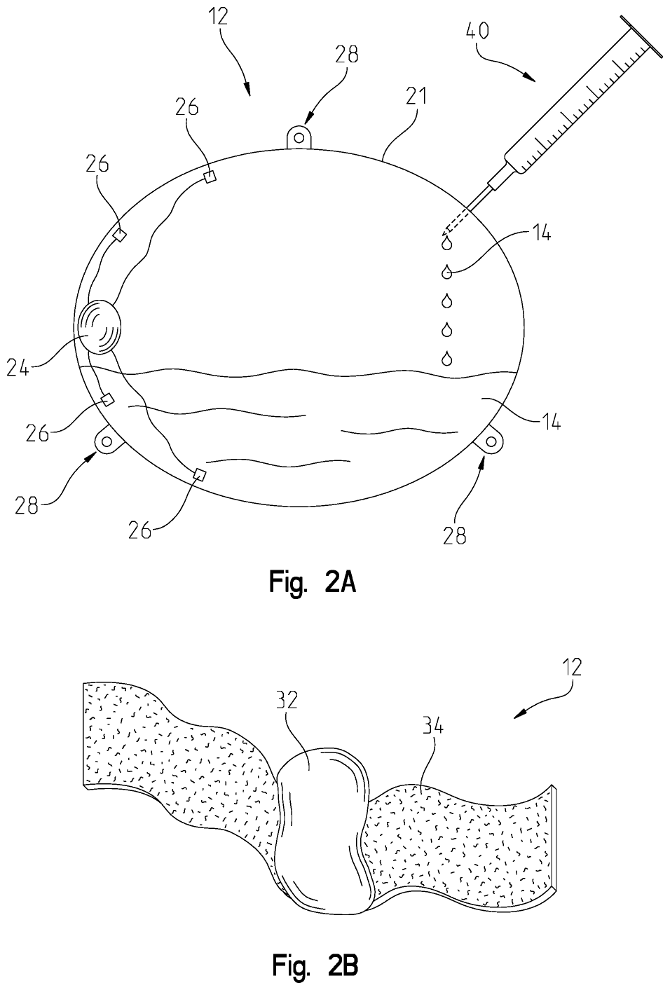

In some instances, the operating physician may use photopolymerization to change the characteristic of the polymer 14. For instance, the polymer 14 may be a photopolymer mixture such as mixture of a light-activated resin or material with one or more additional materials/components. The additional materials may include, but are not limited to, crosslinking agents, photo-initiators, and/or mixtures of other silicones. Additionally, and/or alternatively, the polymer 14 may just include the photopolymer. When exposed to a light source (e.g., in the ultraviolet (UV), infrared (IR), or visible region of the electromagnetic spectrum), the photopolymer 14 may change characteristics such as by increasing in viscosity. In other words, after the permanent implant 12 has been inserted within an individual's breast, the operating physician may inject and fill the permanent implant 12 with a photopolymer 14. The photopolymer 14 may be in a liquid state. After reaching a certain volume/size, the operating physician may perform a process such as by turning on a light source within the permanent implant 12. The light source may emit light (e.g., UV or visible light) onto the photopolymer 14, which causes the photopolymer 14 to change its characteristics including increasing the viscosity of the photopolymer 14. By using photopolymerization within the permanent implant 12, a second surgery is not required to remove this implant 12 and as such, the implant 12 is permanent. Additionally, and/or alternatively, by changing the viscosity of the photopolymer 14 after injection into the permanent implant 12, the photopolymer 14 may be easily injectable into the permanent implant 12 and feel more like actual body tissue. FIG. 2a will describe this in more detail.

FIG. 2a is an exemplary permanent implant 12 in accordance with one or more examples of the present application. The permanent implant 12 may have an outer enclosing 21. The outer enclosing 21 may comprise a plastic lining that encloses or envelopes the permanent implant 12. The outer enclosing 21 may further include one or more tissue securing suture tabs 28. The tissue securing suture tabs 28 may be used to secure the permanent implant 12 within the breast region 10 of the individual.

The injector 40 may be an injection device that includes a storage compartment to store the polymer 14, a release mechanism, and a puncturing mechanism. For example, the injector 40 may be a needle such as a hypodermic needle. The injector 40 may puncture the skin of the individual and the outer enclosing 21 of the permanent implant 12. Then, the injector 40 may use the release mechanism to release the polymer 14 into the permanent implant 12.

The permanent implant 12 may further include one or more light sources 26 and an activation device 24. For example, the activation device 24 may receive instructions from a user device such as a smartphone. The instructions may indicate for the activation device 24 to activate (e.g., turn on) the light sources 26 such that the light sources 26 emit light to cause the polymer 14 to undergo photopolymerization. In other words, the operating physician may provide user input to the user device indicating for the light sources 26 to emit light. The user device may provide instructions to the activation device 24 via a communication protocol (e.g., WI-FI, BLUETOOTH, and so on). In response to the instructions, the activation device 24 may turn on the light sources 26 so the light sources 26 may begin emitting light. This causes the polymer 14 to undergo photopolymerization, which changes the characteristics (viscosity) of the polymer 14.

The user device may be and/or include, but is not limited to, a desktop, laptop, tablet, mobile device (e.g., smartphone device, or other mobile device), smart watch, an internet of things (IOT) device, or any other type of computing device that generally comprises one or more communication components, one or more processing components, and one or more memory components.

The one or more light sources 26 may be any type of light source or emitter that is capable of emitting light. As described above, the light may be in the UV spectrum or in the visible spectrum. In some variations, the light source 26 may be a UV micro-light emitting diode (LED). As shown, the light sources 26 may be operatively coupled to the activation device 24 using a wired connection. Additionally, and/or alternatively, the activation device 24 may be in communication with the light sources 106 using a wireless connection.

The activation device 24 may include a power source such as a power source (e.g., battery), one or more processors, and/or a communications interface. The power source may be a battery and may provide power to the light sources 26 so the light sources 26 may emit light. The processor may be any type of hardware and/or software logic, such as a central processing unit (CPU), RASPBERRY PI processor/logic, controller, and/or logic, that executes computer executable instructions for performing the functionalities described herein. The activation device 104 may use the communications interface to communicate with the user device. For example, the communications interface may be used to receive and/or provide information including receiving instructions to turn on the light sources 26.

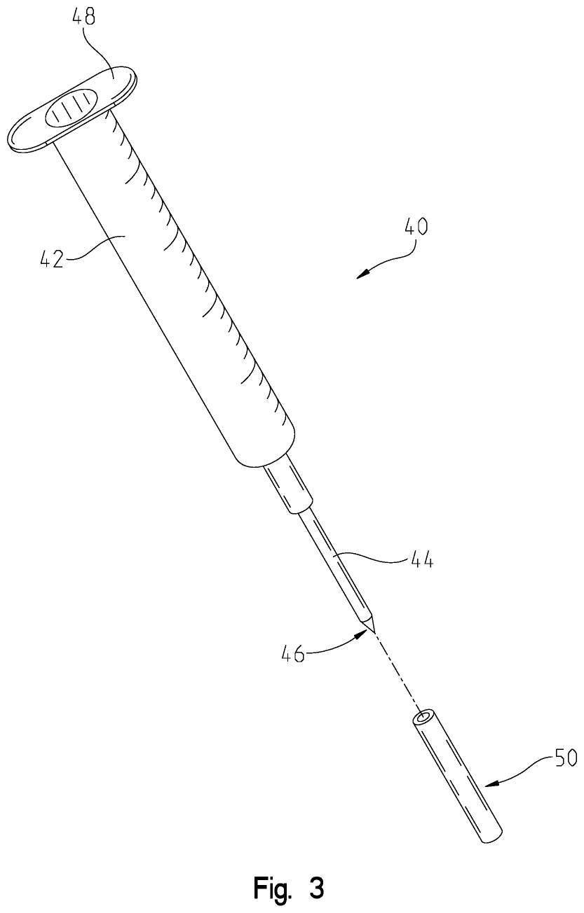

In some variations, the activation device 24 and/or the light sources 26 may be installed in the permanent implant 12 after the permanent implant 12 has been placed within the individual's body. For example, initially, the operating physician may operate on the individual and place the permanent implant 12, without the activation device 24 and/or the light sources 26, into the individual's body. Then, after a period of time, the operating physician may insert the activation device 24 and/or the light sources 26 into the permanent implant 12. For example, the operating physician may use the injector 40 for this purpose as well as for injecting the permanent implant 12 with the polymer 14. FIG. 3 describes this in more detail.

FIG. 2b is another view of the permanent implant 12 showing one or more layers of the outer enclosing 21 in accordance with one or more examples of the present application. In particular, the outer enclosing 21 of the permanent implant 12 may include one or more layers. For example, the inner layer 34 of the outer enclosing 21 may be an inner-reflective layer that is able to reflect light emitted from the light sources 26. For instance, by using the inner-reflective layer 34, additional light may be reflected and provided to the polymer 14 for photopolymerization. The outer enclosing 21 may be enveloped or enclosed over the filling cavity 32, which may be a container that contains the injected polymer 14. In some instances, the outer enclosing 21 may include one layer (e.g., the inner layer 34 that is reflective). In other instances, the outer enclosing 21 may include the inner layer 34 and one or more additional layers. The one or more additional layers may be made of a material different from the inner layer 34.

FIG. 3 is an exemplary injector 40 that injects polymers (e.g., polymer 14) and/or other components (e.g., the activation device 24 and/or the light sources 26) into the permanent implant 12 in accordance with one or more examples of the present application. For example, the injector 40 includes a release mechanism 48 and a storage compartment 42. In operation, the release mechanism 48 may used to fill the storage compartment 42 with polymer 14. Subsequently, the release mechanism 48 may be used to displace the polymer 14 into the permanent implant 12. Additionally, and/or alternatively, the activation device 24 and/or the light sources 26 may be stored within the storage compartment 42. Then, the release mechanism 48 may be used to place the activation device 24 and/or the light sources 26 within the permanent implant 12.

The injector 40 also includes a puncturing mechanism 44. The exterior 50 of the puncturing mechanism 44 may be made out of a metal (e.g., a metal cannula exterior). The interior of the puncturing mechanism 44 may be made out of rubber (e.g., a rubberized interior cannula). The tip 46 of the puncturing mechanism 44 may be a trochar that functions to permit the escape of the polymer 14 from the injector 40 into the permanent implant 12. In operation, the puncturing mechanism 44 may include one or more of the light sources 26 that may be placed within the permanent implant 12.

Additionally, and/or alternatively, the injector 40 may be used to provide power to the light sources 26 and/or provide light to the polymer 14. For instance, the injector 40 may be used in conjunction or as a back-up to the activation device 24 and/or light sources 26. In other words, the trochar 46 may include one or more light emitters (e.g., electrodes) and the metal cannula exterior may provide power to the light emitters within the trochar 46. For example, the activation device 24 may be the primary power source, but may be malfunctioning. In such examples, the injector 40 may provide and/or emit light for the polymer 14 to undergo photopolymerization. In other examples, the activation device 24 may still be operating and the injector 40 may provide additional light to the polymer 14. In yet other examples, the injector 14 may provide additional and/or alternative power to the light sources 26 such that the light sources 26 are able to emit light.

In some variations, the injection of the polymer 14 into the permanent implant 12 may be subsequent to one or more injections of saline into the permanent implant 12. For example, initially, after performing surgery to place the permanent implant 12 into individual's breast, the operating physician may inject saline into the permanent implant 12 to expand the permanent implant 12. Once the permanent implant 12 reaches a certain size, the operating physician may remove the saline and then inject the polymer 14. Then, as described above, the operating physician may perform a process (e.g., photopolymerization) to change a characteristic of the polymer 14.

A number of implementations have been described. Nevertheless, it will be understood that additional modifications may be made without departing from the scope of the inventive concepts described herein, and, accordingly, other examples are within the scope of the following claims. For example, it will be appreciated that the examples of the application described herein are merely exemplary. Variations of these examples may become apparent to those of ordinary skill in the art upon reading the foregoing description. The inventor expects skilled artisans to employ such variations as appropriate, and the inventor intends for the application to be practiced otherwise than as specifically described herein. Accordingly, this application includes all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

It will further be appreciated by those of skill in the art that the execution of the various machine-implemented processes and steps described herein may occur via the computerized execution of processor-executable instructions stored on a non-transitory computer-readable medium, e.g., random access memory (RAM), read-only memory (ROM), programmable read-only memory (PROM), volatile, nonvolatile, or other electronic memory mechanism. Thus, for example, the operations described herein as being performed by computing devices and/or components thereof may be carried out by according to processor-executable instructions and/or installed applications corresponding to software, firmware, and/or computer hardware.

The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the application and does not pose a limitation on the scope of the application unless otherwise claimed. No language in the specification should be construed as indicating any non-claimed element as essential to the practice of the application.

1. A method for performing a breast implant procedure using a permanent expandable prosthetic breast implant, comprising:
subsequent to placing the permanent expandable prosthetic breast implant within a breast region of an individual, injecting a polymer into the permanent expandable prosthetic breast implant; and
changing a viscosity of the polymer within the permanent expandable prosthetic breast implant using a chemical reaction.

2. The method of claim 1, wherein the polymer comprises a photopolymer, and wherein changing the viscosity of the polymer comprises:
activating one or more light sources within the permanent expandable prosthetic breast implant to increase the viscosity of the photopolymer.

3. The method of claim 2, wherein the polymer is a mixture that comprises the photopolymer and at least one other material.

4. The method of claim 2, wherein activating the one or more light sources comprises:
receiving, from a user device and by an activation device within the permanent expandable prosthetic breast implant, one or more instructions indicating to change the viscosity of the polymer; and
in response to receiving the one or more instructions, providing one or more commands to the one or more light sources to emit light within the permanent expandable prosthetic breast implant.

5. The method of claim 4, wherein the activation device comprises a power source, a wireless communications interface, and one or more processors.

6. The method of claim 4, wherein the one or more light sources are inserted within the permanent expandable prosthetic breast implant subsequent to placing the permanent expandable prosthetic breast implant within the breast region of the individual.

7. The method of claim 6, wherein the activation device is inserted within the permanent expandable prosthetic breast implant subsequent to placing the permanent expandable prosthetic breast implant within the breast region of the individual.

8. The method of claim 4, wherein the one or more light sources is configured to emit light in the ultraviolet (UV) spectrum or visible spectrum.

9. A permanent expandable prosthetic breast implant, comprising:
an exterior surface layer;
an interior spacing within the exterior surface layer, wherein the interior spacing comprises a photopolymer; and
one or more light sources within the exterior surface layer, wherein the one or more light sources are configured to cause a change in a characteristic of the photopolymer by emitting a light.

10. The permanent expandable prosthetic breast implant of claim 9, wherein the permanent expandable prosthetic breast implant further comprises:
an activation device configured to:
receive, from a user device, an instruction to change in the characteristic of the photopolymer; and
in response to receiving the instruction, providing one or more commands to the one or more light sources to emit the light.

11. The permanent expandable prosthetic breast implant of claim 10, wherein the activation device comprises a power source, a wireless communications interface, and one or more processors.

12. The permanent expandable prosthetic breast implant of claim 10, wherein the one or more light sources are inserted within the permanent expandable prosthetic breast implant subsequent to placing the permanent expandable prosthetic breast implant within a breast region of an individual.

13. The permanent expandable prosthetic breast implant of claim 10, wherein the activation device is inserted within the permanent expandable prosthetic breast implant subsequent to placing the permanent expandable prosthetic breast implant within a breast region of an individual.

14. The permanent expandable prosthetic breast implant of claim 9, wherein the light is in an ultraviolet (UV) spectrum.

15. The permanent expandable prosthetic breast implant of claim 9, wherein the light is in a visible spectrum.

16. A system, comprising:
a permanent expandable prosthetic breast implant, the permanent expandable prosthetic breast implant comprising:
an exterior surface layer;
an interior spacing within the exterior surface layer, wherein the interior spacing comprises a photopolymer; and
one or more light sources within the exterior surface layer, wherein the one or more light sources are configured to cause a change in a characteristic of the photopolymer by emitting a light; and
a user device configured to provide an instruction to cause the change in the characteristic of the polymer.

17. The system of claim 16, wherein the permanent expandable prosthetic breast implant further comprises:
an activation device configured to:
receive, from the user device, the instruction to cause the change in the characteristic of the photopolymer; and
in response to receiving the instruction, providing one or more commands to the one or more light sources to emit the light.

18. The system of claim 17, wherein the activation device comprises a power source, a wireless communications interface, and one or more processors.

19. The system of claim 16, wherein the light is in an ultraviolet (UV) spectrum.

20. The system of claim 16, wherein the light is in a visible spectrum.
